(19) United States
(12) Patent Application Publication
(10) Pub. No.: US 2012/0302880 A1
(43) Pub. Date: Nov. 29, 2012

(54) SYSTEM AND METHOD FOR SPECIFICITY-BASED MULTIMODALITY THREE- DIMENSIONAL OPTICAL TOMOGRAPHY IMAGING

(75) Inventors: Jie TIAN, Beijing (CN); Xin YANG, Beijing (CN); Kai LIU, Beijing (CN); Dong HAN, Beijing (CN); Chenghu QIN, Beijing (CN)

(73) Assignee: Institute of Automation, Chinese Academy of Sciences, Beijing (CN)

(21) Appl. No.: 13/535,774

(22) Filed: Jun. 28, 2012

Related U.S. Application Data
(63) Continuation of application No. PCT/CN2010/001930, filed on Nov. 30, 2010.

(51) Int. Cl. A61B 6/02; A61B 6/03

(52) U.S. Cl. 600/427

(57) ABSTRACT

A system and method for specificity-based multimodality three-dimensional optical tomography imaging comprises steps of: optical imaging to obtain a light intensity of body surface optical signal of an imaging target; CT imaging to obtain structure volume data; establishing an equation representing a linear relationship between the distribution of the obtained light intensity of body surface optical signal of the imaging target, the obtained CT discrete mesh data and the distribution of unknown internal self-luminescence light sources; establishing a dynamic sparse regularization target function in every iteration for the equation; and reconstructing a tomography image. The present invention well considers the optical specificity of tissue, in which there is a non-uniform optical characteristic parameter distribution within the same tissue when finite element modeling is used, which is closer to the real situation, so that an accurate imaging effect is achieved.

### BACKGROUND OF THE INVENTION

1. Field of the Invention

The present invention relates to an imaging system, more particularly to a system and method for specificity-based multimodality three-dimensional optical tomography imaging.

2. Description of Prior Art

Recently, optical molecular image is a new technology developed fast among various modes of molecular image. The optical molecular image technology may apply a successive on-body imaging to the entire of an organism in a noninvasive manner in real time, and visualizes variable information such as physiological, metabolism, or cell molecule level of the organism by using a method of three-dimensional tomography imaging, facilitating the development of related biomedical research applications.

Three-dimensional optical tomography imaging is an ill-posed inverse problem due to the limited information that may be measured during the imaging process to locate a target to be reconstructed, and thus there is no unique finite solution for such inverse problem in general. In order to get a reasonable result, it is desirable to apply more known information and constraint conditions in the construction to mitigate ill-posedness of the problem. Currently, the widely used approaches include multi-spectral boundary data measuring and permissible source region setting. Although these approaches improve the reliability of the tomography imaging to a certain degree, they impose critical requirement on the experiment conditions and is hard to be located accurately in practical imaging applications.

The robustness of three-dimensional optical tomography imaging also relies on the development of a new imaging technology. Most of the traditional methods are local optimal in the view of optimization, so that the process of imaging highly depends on an iteration initial guess. Accordingly, it is necessary to provide a sufficiently precise initial guess and performs the reconstruction in a quite small area to achieve an ideal imaging effect, and consequentially the practicability of the imaging technology is reduced. In the process of image reconstructing, the imaging quality also depends on a parameter setting, which always depends on only an experiential selection. These limitations seriously constrain the application of optical three-dimensional imaging tomography.

### SUMMARY OF THE INVENTION

For the above described problems, an object of the present invention is to provide a system and method for specificity-based multimodality three-dimensional optical tomography imaging.

In accordance with an aspect of the present invention, a method for specificity-based multimodality three-dimensional optical tomography imaging comprises steps of:

optical imaging to obtain a light intensity of body surface optical signal of an imaging target;
CT imaging to obtain structure volume data;
establishing an equation representing the linear relationship between the distribution of the obtained light intensity of body surface optical signal of the imaging target, the obtained CT discrete mesh data and the distribution of unknown internal self-luminescence light sources;
establishing a dynamic sparse regularization target function in every iteration for the equation; and
reconstructing a tomography image.

In accordance with another aspect of the present invention, a system for specificity-based multimodality three-dimensional optical tomography imaging comprises:

an optical imaging sub-module for obtain a light intensity of body surface optical signal of an imaging object;
a CT imaging sub-module for obtaining structure volume data of the imaging object;
a translating table for controlling the back and forth movements of the imaging object;
a rotating table for rotating to perform optical multi-angle imaging and CT cone beam X-ray scanning on the imaging object;
an electronic control system for controlling the translating table and rotating table;
a rotation control and processing software platform for establishing an equation representing the linear relationship between the distribution of the obtained light intensity of body surface optical signal of the imaging target, the obtained CT discrete mesh data and the distribution of unknown internal self-luminescence light sources, establishing a dynamic sparse regularization target function in every iteration for the equation, and
reconstructing a tomography image.

The present invention well considers the optical specificity of tissue, in which there is a non-uniform optical characteristic parameter distribution within the same tissue when finite element modeling is used, which is closer to the real situation, so that an accurate imaging effect is achieved. The reconstruction method of the present invention may apply a whole-body three-dimensional tomography imaging to the imaging object, avoiding the dependence on the priori knowledge of locating a rough distributed position of the reconstruction target. The invention uses the sparse regularization technology, which improves the robustness of image reconstruction by using the sparse distribution characteristic of the reconstruction target within the imaging object, and greatly reduces the dependence on the regularization parameter selection.

### BRIEF DESCRIPTION OF THE DRAWINGS

FIG. 1 is a block diagram of the hardware part of the multimodality imaging in accordance with the present invention.

FIG. 2 is an overall flow chart of the implementation of the specificity-based multimodality three-dimensional optical tomography system in accordance with the present invention.

FIG. 3 is a flow chart of obtaining the discrete volume data in accordance with the present invention.

FIG. 4 is a flowchart of the implementation of the tomography image reconstruction module in accordance with the present invention.

FIG. 5 is a diagram showing an imaging result of the CT sub-module in the multimodality optical three-dimensional tomography imaging system.

FIG. 6 is a diagram showing multi-angle imaging in the optical imaging sub-module of the multimodality optical three-dimensional tomography imaging system.

FIG. 7 shows a specificity model used for the imaging object in an embodiment.

FIG. 8 is a diagram showing tomography imaging results under different regularization parameters.

FIG. 9 is a diagram showing tomography imaging results under different initial iteration values.

### DETAILED DESCRIPTION OF THE PREFERRED EMBODIMENTS

In order to solve the ill-posedness problem in reconstruction, a method for optical three-dimensional tomography imaging based on a multimodality combination technology is provided in the present invention. The present invention involves mainly two modes: optical imaging and X-ray tomography imaging (CT). On one hand, optical imaging has an advantage of high contrast, but its spatial resolution is poor; on the other hand, X-ray tomography imaging (CT) has a high spatial resolution, but its contrast is poor. Therefore, combination of these two modes can effectively improve the quality of imaging and provide more comprehensive physiological information, achieving a complementary of advantages. In particular, the CT imaging technology and the optical imaging technology is combined, and more independent information are introduced to the image reconstruction for optical three-dimensional tomography imaging by providing the knowledge of the complex surface figure and internal anatomical structure of the imaging object, such that the ill-posedness in the imaging of the imaging object is mitigated, thereby the accuracy and reliability of the imaging are improved.

After the anatomical structure information is obtained by the CT imaging technology, it is also desirable to take further research on how to make full use of such structure information. An intuitive manner is to assume optical parameters in the imaging object are homogeneous, which means that optical parameters in the same tissue are consistent. In general, this assumption is a reasonable estimation of the real situation in case that there is no more priori knowledge. However, in many cases, such assumption of homogeneous has a great error, for example, when imaging a tumor, optical absorption coefficient in tumor area is higher than that in the surrounding normal tissue area due to the existence of newly formed blood vessels. Accordingly the distribution of optical parameters is not uniform even in the same tissue, i.e. the biological tissue has specificity. Therefore, the present invention provides a specificity-based optical tomography imaging technology, which can model an optical characteristic of a tissue more accurately and thus achieve a more accurate imaging result.

In order to deal with the robustness problem of optical three-dimensional tomography imaging, the present invention provides a method for reconstructing based on whole-body imaging without priori knowledge of the position of the reconstruction target; and a global optimization method is used to greatly reduce the dependency on the initial value. In addition, the present invention uses a sparse regularization technique to makes full use of the sparseness characteristics of the reconstruction target, increasing the robustness of imaging and greatly decreasing the dependency on the regularization parameter selection.

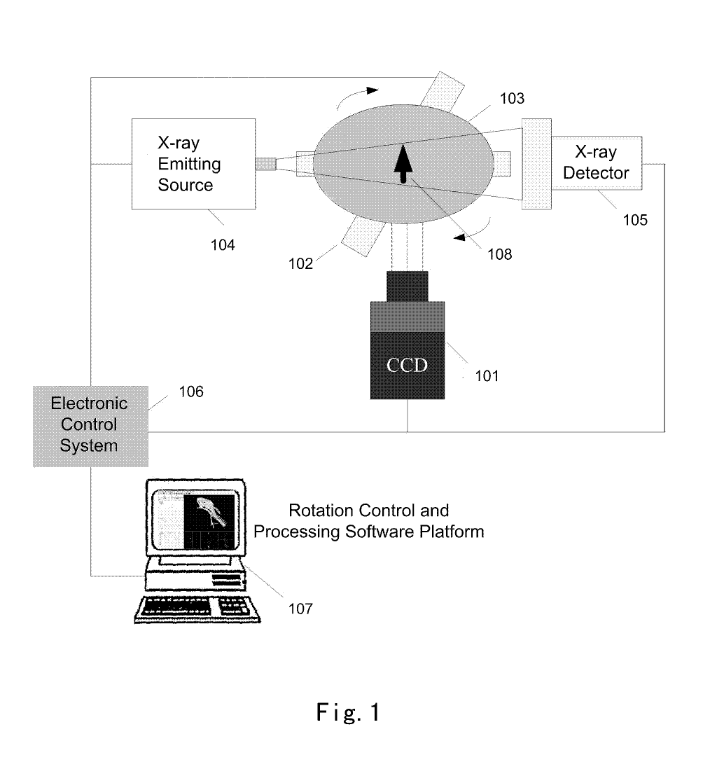

As shown in FIG. 1, the hardware part of the multimodality imaging of the present invention comprises multimodality modules of two modes (optical mode and CT mode) and their control and processing software platform. The optical imaging sub-module comprises a cryogenic cooled CCD device 101 (including a lens and a CCD camera), an imaging two-dimensional translating table 102 driven by a step motor, a rotating table 103, and an electronic control system 106, wherein the translating table, the rotating table and the electronic control systems are shared by the two imaging sub-modules. The optical imaging sub-module and the CT imaging sub-module are perpendicular to each other, such that the two modules may collect signals simultaneously. Such imaging structure on one hand can shorten the imaging time, and on the other hand can increase the matching accuracy between the surface fluorescence information and the anatomical structure information, thereby improving the accuracy of the reconstruction of the light source. The lens of the CCD device 101 has a numerical aperture and the CCD camera is cooled by liquid nitrogen down to −110° C. to reduce dark current noise and improve the signal to noise ratio of the detected light intensity signal, wherein the data collected by the CCD camera is the fluorescence data of the surface of the imaging object, and will be used as known measurement data in the reconstruction process of the light source. The imaging two-dimensional translating table 102 and the rotating table 103 are driven by the stepper motor drive. The translating table is controlled by the electronic control system 106. By position adjustment using the imaging two-dimensional translation 102, the vertical central axis of the imaging object 108 is ensured to coincide with the axis of the rotary table, while the imaging object may be controlled to move back and forth in accordance with the requirements of the imaging size. The rotating table 103 is controlled by the electronic control system 106 to rotate in a stepping manner, achieving a multi-angle X-ray projection data collection for the CT imaging module and a multi-angle surface fluorescence signal collection for the optical imaging module, thereby increasing the amount of known measurement data, mitigating the ill-posedness of the reconstruction problem, and increasing the accuracy of the reconstruction of the light source. The CT imaging sub-module comprises an X-ray emitting source 104, an X-ray detector 105. The CT imaging sub-module uses the X-ray of the X-ray emitting source 104 to radiate an X-ray having certain energy to the imaging object. The rotating table is rotated to achieve multi-angle projection data collection. X-ray collection is accomplished by the X-ray detector 104. By CT image reconstruction and discretization of the reconstruction result, accurate tetrahedral mesh data may be provided for the reconstruction of fluorescent light source. The rotation control and processing software platform 107 for establishing an equation representing the linear relationship between the distribution of the obtained light, intensity of body surface optical signal of the imaging target, the obtained CT discrete mesh data and the distribution of unknown internal self-luminescence light sources, establishing a dynamic sparse regularization target function in every iteration for the equation, and reconstructing a tomography image, comprises a module for controlling the image collection, a module for segmenting image, reducing noise, selecting area of interest, and CT image constructing, wherein the image collection and control module is responsible for sending an instruction to the electronic control system 106 to control the movement of the rotating and translating tables and the collection of the X-ray and the fluorescence signal; the function of the module for segmenting image, reducing noise, selecting area of interest is to extract useful fluorescence signal from the background noise to improve signal to noise ratio, achieving a more accurate reconstruction result of the light source; the CT reconstruction module is responsible for using multi-angle X-ray projection data to reconstruct the anatomical structure information, and the reconstructed data may be mesh discretized to assist the reconstruction of the fluorescent light source.

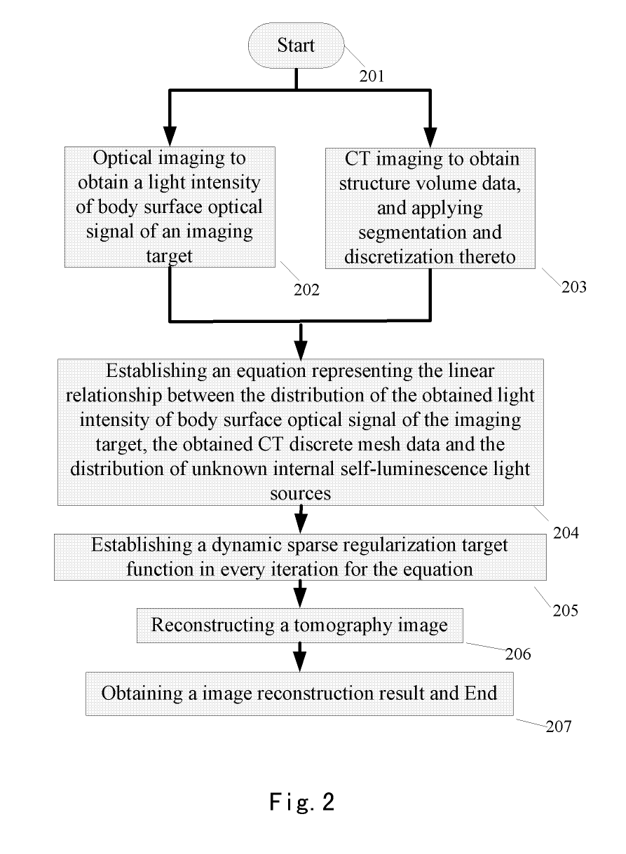

FIG. 2 is an overall flow chart of the implementation of the system for specificity-based multimodality optical three-dimensional tomography imaging in accordance with the present application.

The process begins with step 201.

In step 202, an imaging object is placed on the imaging two-dimensional translating table and rotating table, the movement, rotation of the imaging object is controlled by the control and processing software platform such that the imaging object may be contained in both the imaging range of the optical imaging sub-module and the imaging range of the CT imaging sub-module; and through controlling the step motor to drive by the control and processing software platform, the optical imaging sub-module is used to apply multi-angle imaging to the body surface of the imaging object to achieve an optical signal distribution of 360° on the body surface.

In step 203, the CT imaging sub-module is used to obtain X-ray image data of the imaging object, and the structure volume data information of the imaging object is reconstructed by the software platform and then is subjected to image segmentation and mesh discretization.

In step 204, a finite element equation, representing a linear relationship between the distribution of the light intensity of body surface optical signal of the imaging target obtained by optical imaging, the CT discrete mesh data obtained by CT imaging, and the distribution of unknown internal self-luminescence light sources, is established based on an approximate model describing the diffusion of the light propagation within the imaging object. The equation is represented as: MX=Φ, where M is a system matrix describing the linear relationship, X is a vector representing the distribution of the reconstruction target within the imaging object, Φ is a vector representing a distribution of light intensity of optical signal on the surface of the imaging object.

In step 205, establishing a target function updated in every iteration. The target function T^{(k)}(X) is typically as follows:

$${T}^{( k )} ( X ) = \frac{1}{2} {‖ MX - Φ ‖}_{2}^{2} + \frac{λ}{2} {‖ {W}_{s}^{( k ) 1 / 2} X ‖}_{2}^{2} + λ ( 1 - \frac{p}{2} ) S ( {X}^{( k )} ) ( k \geq 0 ) ,$$

where |MX−Φ∥²₂ represents a precision item, ∥W_{S}^{(k)1/2}X∥₂² is a sparse regularization item, and

$$( 1 - \frac{p}{2} ) S ( {X}^{( k )} )$$

ensures the target function in every regularization iteration is equivalent to a target function

$$F ( X ) = \frac{1}{2} {‖ MX - Φ ‖}_{2}^{2} + \frac{λ}{2} {‖ X ‖}_{p}^{p} ,$$

where the sparse weight matrix W_{S}^{(k)}=diag(τ_{S,ε}_{_{S}}(X^{(k)})), diag(□) represents diagonal matrix, ε_{S} represents a weight matrix threshold, and τ_{S,ε}_{_{S}}(χ) is expressed as:

$${τ}_{S , {ɛ}_{S}} ( x ) = \{ \begin{matrix}{\vert x \vert}^{p - 2} & if \vert x \vert > {ɛ}_{S} \\ 0 & if \vert x \vert \leq {ɛ}_{S}\end{matrix}$$

In step 206, tomography imaging is performed by using the three-dimensional tomography imaging reconstruction method.

In step 207, a reconstruction result is obtained and the process is ended.

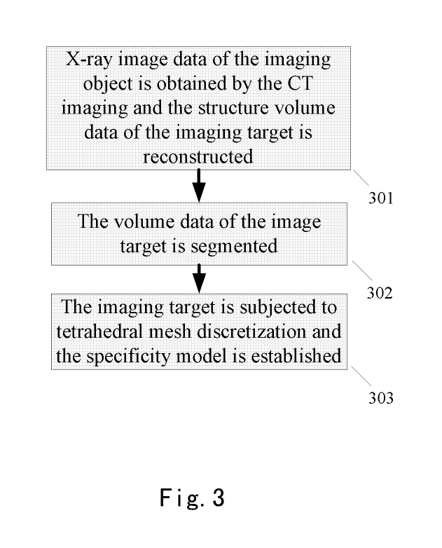

As shown in FIG. 3, in step 301, X-ray image data of the imaging object is obtained by the CT imaging sub-module and the structure volume data of the imaging target is reconstructed by the software platform.

In step 302, the CT data information is segmented by the software platform to obtain a distribution map of the tissues of a primary organ and form a surface mesh.

In step 303, a tetrahedron mesh is formed by using surface mesh of respective tissues, and then non-uniform optical characteristic parameters are assigned to the tetrahedron based on a specificity model.

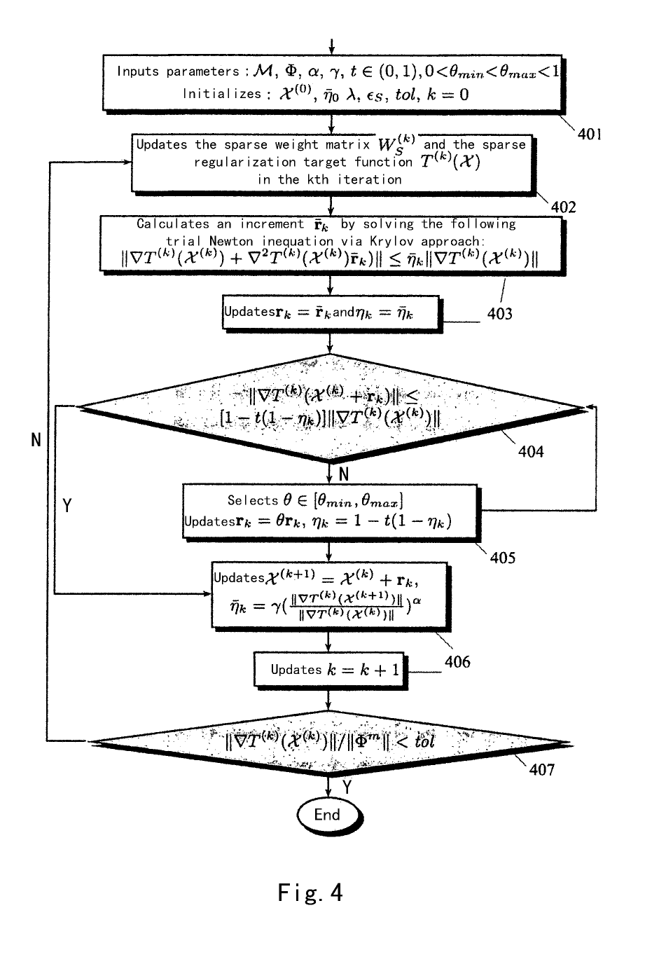

As shown in FIG. 4, the tomography imaging of the present invention is implemented as follows.

In step 401, inputs the system matrix M, the surface measured optical vector Φ, an exponential gain coefficient α, the weight gain coefficient γ, the maximum θₘₐₓ and minimum θₘᵢₙ of attenuation coefficient, and then initializes the distribution vector X⁽⁰⁾ of an unknown reconstruction target, the sparse weight matrix W_{S}⁽⁰⁾, a reconstruction termination threshold η̅₀, the regularization parameter λ, the weight matrix threshold value ε_{S} and an iteration termination threshold tol, and sets an initial number of iterations as k=0;

In step 402, updates W_{S}^{(k)}=diag(τ_{S,ε}_{_{S}}(X^{(k)})) and the sparse regularization target function T^{(k)}(X) in the k^{th} iteration.

In step 403, calculates an increment r̅k of the reconstruction target distribution vector by using the following in equation:

∥∇T^{(k)}(X^{(k)})+∇²T^{(k)}(X^{(k)}) r̅k∥≦ η̅ₖ∥∇T^{(k)}(X^{(k)}))∥, and

sets the increment of reconstruction target rₖ= r̅k and the reconstruction termination threshold ηₖ= η̅ₖ, where ∇T^{(k)} is a gradient of the target function in the k^{th} iteration: ∇T^{(k)}=(M^{T}M+λW_{S}^{(k)})X−M^{T}Φ, and ∇²T^{(k)} is a Hessen matrix of the target function in the k^{th} iteration: ∇²T^{(k)}=M^{T}M+λW_{S}^{(k)}.

In step 404, determines whether rₖ meets the following in equation:

∥∇T^{(k)}(X^{(k)}+rₖ)∥≦[1−t(1−ηₖ)]∥∇T^{(k)}(X^{(k)})∥, and

if not, turns to step 405, otherwise, turns to step 406;

In step 405, selects θε(θₘᵢₙ, θₘₐₓ), updates rₖ=θrₖ, ηₖ=1−t(1−ηₖ), and skips to step 404.

In step 406, updates the reconstruction target distribution vector X^{(k+1)}=X^{(k)}+rₖ, calculates η̅ₖ=γ(∇T^{(k)}(X^{(k+1)})/∇T^{(k)}(X^{(k)}))^{α}, and updates the number of iteratins k=k+1.

In step 407, determines whether the in equation

∥∇T^{(k)}(X^{(k)})∥/∥Φ∥<tol

fulfilled, and,
if not, turns to step 402, otherwise, terminates the image reconstruction.

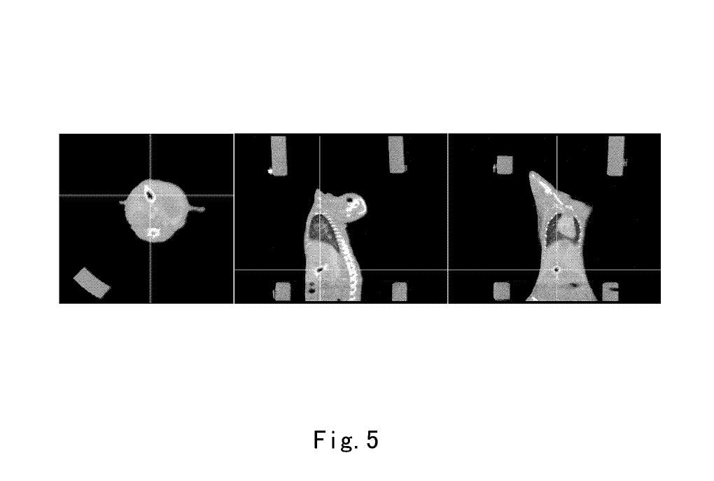

FIG. 5 shows imaging results of transverse section, sagittal section and coronal section by the CT imaging sub-module in the multimodality imaging system. The scanning voltage of the X-ray source is 50 kV, the power is 50 W, the integration time of the detector is 0.467 s, the speed of rotating table is 1.0°/s, the single-frame projected image size is 1120×2344, the single-frame imaging time is 3.0 s, and the number of projections is 360. An aluminum plate having a thickness of 0.5 mm is used to filter out the soft X-ray to increase the signal to noise ratio. Based on CT imaging, the position of the reconstruction target may be located as (25.54 21.31 8.52).

After the data collection is completed, three-dimensional volume data can be reconstructed by the control and processing software platform, in which the voxel size is 0.10×0.10×0.20 (transverse section×sagittal section×coronal section).

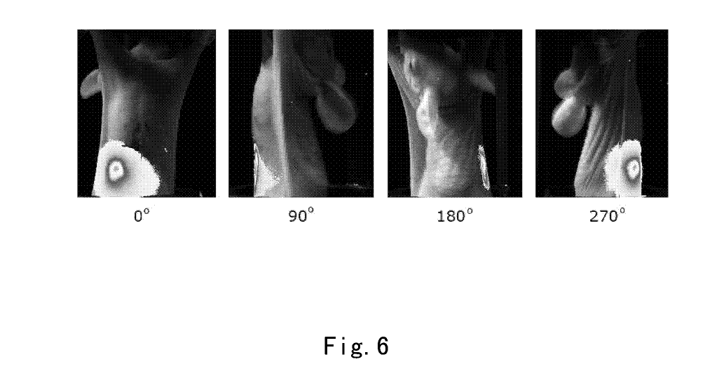

FIG. 6 shows a multi-angle imaging result of the imaging object by the optical imaging sub-module. Before imaging, the CCD is cooled to −110° C. In this optical imaging, exposure time of CCD is 60 sec, aperture f is 2.8, focal length is 55 mm, the distance between the imaging object and the lens is 15 cm. The speed of rotating table is 1.5°/s. The imaging object is fixed on the rotating table, to obtain the light intensity distribution of the imaging object at various angles. The rotating table rotates clockwise, and the CCD images the imaging object each time the rotating table rotates 90°. The acquired imaging pixels are incorporated, i.e. four pixels are incorporated into one pixel. Then, the imaging map is overlaid with the white light map of the imaging object to locate the two-dimensional position of the reconstruction target roughly.

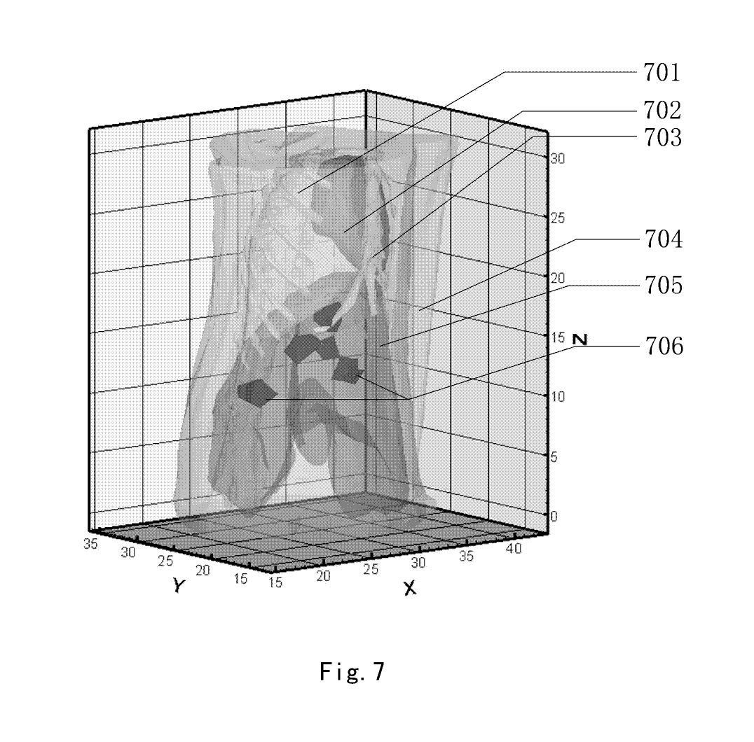

As shown in FIG. 7, based on the volume data obtained by the aforementioned CT imaging, data is segmented into primary organs and tissues with different properties within the organs and the entire volume data is subjected to tetrahedral discretization. Firstly, interactively segment is applied to the heart, lung, liver and internal tissue therein in transverse section, then skeletons is extracted by using an automatic segmentation method, and the rest is considered as muscle. A gray value is set for each portion to synthesize into data of whole body. Next, the volume data is subjected to tetrahedral discretization. Firstly, a surface mesh of an interface between different portions of the volume data is obtained, then a volume mesh is divided after the surface mesh is simplified, and finally a discretized mesh is obtained. The discretized mesh is composed of 23752 tetrahedrons and 4560 nodes with 1092 nodes on the outer surface. In FIG. 7, 701 denotes lung, 702 denotes heart, 703 denotes skeletons, 704 denotes muscle, 705 denotes liver, 706 denotes the dark region in liver which indicate that there is non-uniform optical parameter in the liver tissue, namely the tissue has specificity.

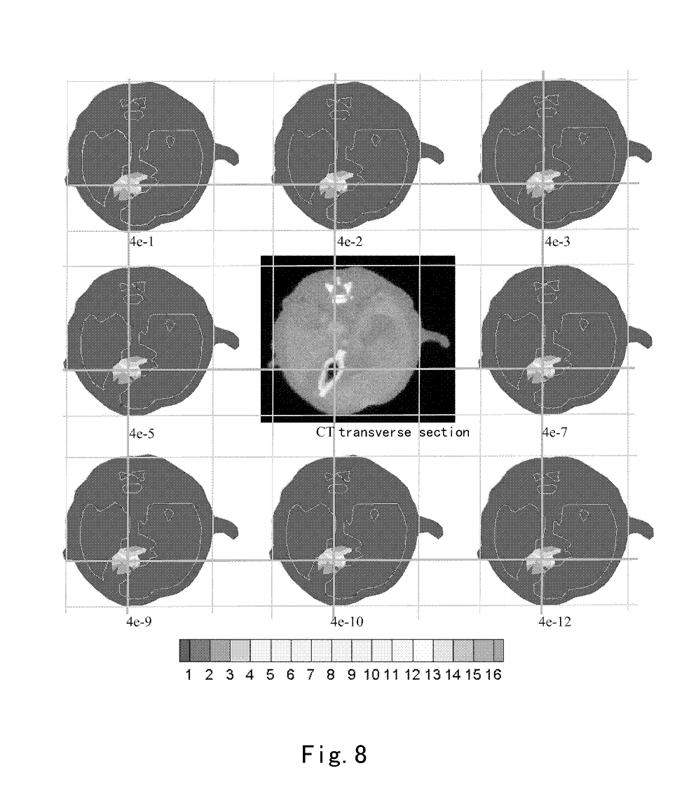

As shown in FIG. 8, based on the optical signal distribution and CT volume data obtained by aforementioned multimodality system and volume mesh data obtained by segmentation and discretization, image reconstruction is performed under different regularization parameter λ.

The input parameters include: system matrix M (1092×4560) and the surface measured optical vector Φ (1092×1). p=1 in the sparse regularization target function. The exponential gain coefficient α=1.618, and the weight gain coefficient γ=0.01, the maximum of attenuation coefficient θₘₐₓ=0.99 and minimum of attenuation coefficient θₘᵢₙ=0.01. Then unknown reconstruction object distribution vector is initialized as homogeneous distribution and X⁽⁰⁾=0, sparse weight matrix W_{S}⁽⁰⁾=I (unit matrix), the resolving threshold η̅₀=10, the weight matrix threshold ε_{S}=0.02 and the iteration termination threshold tol=0.2, set k=0. The regularization parameter λ is set as 4×10⁻¹, 4×10⁻², 4×10⁻³, 4×10⁻⁵, 4×10⁻⁷, 4×10⁻⁹, 4×10⁻¹⁰, 4×10⁻¹² respectively. The difference between the maximum and minimum of the regularization parameter λ is of the order of magnitude of 11.

The method for image reconstructing based on sparse regularization and entire body imaging in accordance with the present invention is used for reconstruction, depending on multimodality optical and CT data, under regularization parameters of different orders of magnitude. The image reconstruction result shows that the reconstruction target within the imaging object is insensitive to the choice of regularization parameter. The reconstruction result is substantially consistent under is different regularization parameters and the reconstruction errors are all within 1 mm.

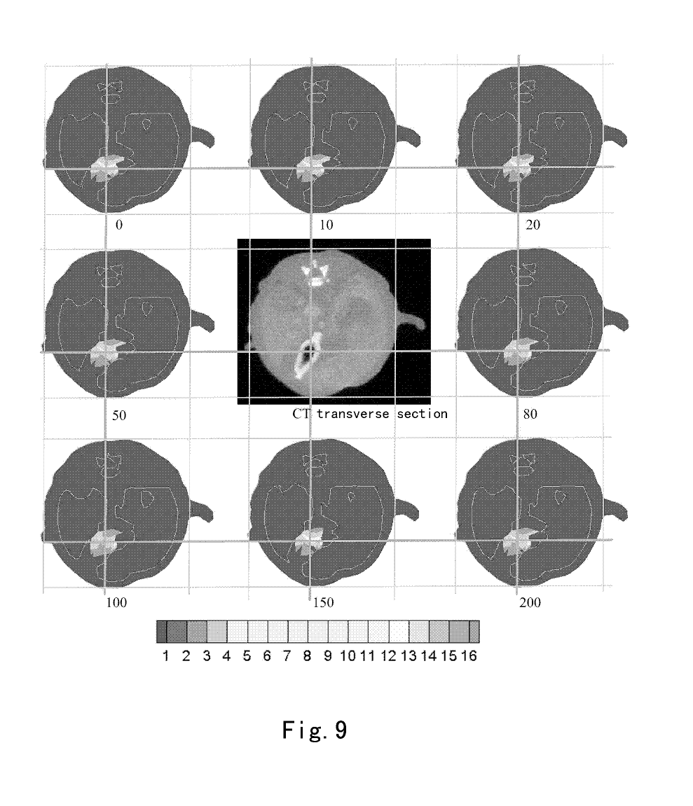

As shown in FIG. 9, based on the optical signal distribution and CT volume data acquired by aforementioned multimodality system and mesh data obtained by segmentation and discretization, image reconstruction is performed under different initial values of distribution of reconstruction targets.

The unknown reconstruction object distribution vector is initialized as homogeneous distribution and adopt the following 8 groups parameters: X⁽⁰⁾=0, X⁽⁰⁾=10, X⁽⁰⁾=20, X⁽⁰⁾=50, X⁽⁰⁾=80, X⁽⁰⁾=100, X⁽⁰⁾=150, X⁽⁰⁾=200. The regularization parameters λ are set to 4×10⁻² respectively, and the other parameters are the same as in FIG. 7.

Likewise, the method for image reconstructing of the present invention is used to reconstruct under above described different initial values, in which the reconstruction result shows that the obtained reconstruction target distribution is substantially consistent with the real position and the reconstruction errors are all within 1 mm.

The present invention can establish a detection technology platform integrating vivo molecular imaging study, medical application and drug screening, on which a robust reconstruction may be performed, providing a foundation for a practical application such as vivo locating of reconstruction target.

The foregoing description gives only the embodiments of the present invention, and the scope of the present invention is not limited thereto. It will be appreciated by those skilled in the art that many modifications and alternatives can be made without departing from the principles and spirits of the invention, and they shall fall into the scope of the present invention. Therefore the scope of the present invention is determined by the claims.

What is claimed is:

1. A method for specificity-based multimodality three-dimensional optical tomography imaging, in which the method comprises steps of:
optical imaging to obtain a light intensity of body surface optical signal of an imaging target;
CT imaging to obtain structure volume data;
establishing an equation representing a linear relationship between the distribution of the obtained light intensity of body surface optical signal of the imaging target, the obtained CT discrete mesh data and the distribution of unknown internal self-luminescence light sources;
establishing a dynamic sparse regularization target function in every iteration for the equation; and
reconstructing a tomography image.

2. The method of claim 1, wherein the optical imaging is a multi-angle imaging of the body surface of an imaging object.

3. The method of claim 1, wherein obtaining structure volume data comprises steps of:
segmenting the structure data of imaging target body; and
forming a tetrahedron mesh by using a surface mesh.

4. The method of claim 3, further comprises assigning non-uniform optical characteristic parameters to the tetrahedron.

5. The method of claim 4, wherein non-uniform optical characteristic parameters are assigned to the tetrahedron based on the specificity model.

6. The method of claim 1, wherein the equation is represented as:
MX=Φ
where M is a system matrix describing the linear relationship, X is a vector representing the distribution of the reconstruction target within the imaging object, Φ is a vector representing a distribution of light intensity of optical signal on the surface of the imaging object.

7. The method of claim 6, wherein the sparse regularization target function T^{(k)}(X):
$${T}^{( k )} ( X ) = \frac{1}{2} {‖ MX - Φ ‖}_{2}^{2} + \frac{λ}{2} {‖ {W}_{s}^{( k ) 1 / 2} X ‖}_{2}^{2} + λ ( 1 - \frac{p}{2} ) S ( {X}^{( k )} ) ( k \geq 0 ) ,$$
is updated in every iteration,
where |MX−Φ∥₂² represents a precision item, |W_{S}^{(k)1/2}X∥₂² is a sparse regularization item, and
$$( 1 - \frac{p}{2} ) S ( {X}^{( k )} )$$
ensures the target function in every regularization iteration is equivalent to a target function
$$F ( X ) = \frac{1}{2} {‖ MX - Φ ‖}_{2}^{2} + \frac{λ}{2} {‖ X ‖}_{p}^{p} ,$$
where the sparse weight matrix W_{S}^{(k)}=diag(τ_{S,ε}_{_{S}}(X^{(k)})), diag(□) represents diagonal matrix, ε_{S} represents a weight matrix threshold, and τ_{S,ε}_{_{S}}(χ) is expressed as:
$${τ}_{S , {ɛ}_{S}} ( x ) = \{ \begin{matrix}{\vert x \vert}^{p - 2} & if \vert x \vert > {ɛ}_{S} \\ 0 & if \vert x \vert \leq {ɛ}_{S}\end{matrix}$$

8. The method of claim 7, wherein reconstructing the tomography image comprises steps of:
1) inputting the system matrix M, the surface measured optical vector Φ, an exponential gain coefficient α, the weight gain coefficient γ, the maximum θₘₐₓ and minimum θₘᵢₙ of attenuation coefficient, and then initializing the distribution vector X⁽⁰⁾ of an unknown reconstruction target, the sparse weight matrix W_{S}⁽⁰⁾, a reconstruction termination threshold η̅₀, regularization parameter λ, the weight matrix threshold value ε_{S} and an iteration termination threshold tol, and setting an initial number of iterations as k=0;
2) updating W_{S}^{(k)}=diag(τ_{S,ε}_{_{S}}(X^{(k)})) and the sparse regularization target function T^{(k)}(X) in the k^{th} iteration;
3) calculating an increment r̅k of the reconstruction target distribution vector by using the following in equation:
∥∇T^{(k)}(X^{(k)})+∇²T^{(k)}(X^{(k)}) r̅k∥≦ η̅ₖ∥∇T^{(k)}(X^{(k)}))∥, and
setting the increment of reconstruction target rₖ= r̅k and the reconstruction termination threshold ηₖ= η̅ₖ, where ∇T^{(k)} is a gradient of the target function in the k^{th} iteration: ∇T^{(k)}=(M^{T}M+λW_{S}^{(k)})X−M^{T}Φ, and ∇²T^{(k)} is a Hessen matrix of the target function in the k^{th} iteration: ∇²T^{(k)}=M^{T}M+λW_{S}^{(k)};
4) determining whether rₖ meets the following in equation:
∥∇T^{(k)}(X^{(k)}+rₖ)∥≦[1−t(1−ηₖ)]∥∇T^{(k)}(X^{(k)})∥, and
if not, turning to step 5), otherwise, turning to step 6);
5) selecting θε(θₘᵢₙ, θₘₐₓ), updating rₖ=θrₖ, ηₖ1−t(1−ηₖ), and skipping to step 4);
6) updating the reconstruction target distribution vector X^{(k+1)}=X^{(k)}+rₖ, calculating η̅ₖ=γ(∇T^{(k)}(X^{(k+1)})/∇T^{(k)}(X^{(k)}))^{α}, and updating the number k of iteratins k=+1;
7) determining whether the in equation
∥∇T^{(k)}(X^{(k)})∥/∥Φ∥<tol
fulfilled, and,
if not, turning to step 2), otherwise, terminating the three-dimensional tomography image reconstruction.

9. A system for specificity-based multimodality three-dimensional optical tomography imaging, comprising:
an optical imaging sub-module for obtain a light intensity of body surface optical signal of an imaging object;
a CT imaging sub-module for obtaining structure volume data of the imaging object;
a translating table for controlling the back and forth movements of the imaging object;
a rotating table for rotating to perform optical multi-angle imaging and CT cone beam X-ray scanning on the imaging object;
an electronic control system for controlling the translating table and rotating table; and
a rotation control and processing software platform for establishing an equation representing the linear relationship between the distribution of the obtained light intensity of body surface optical signal of the imaging target, the obtained CT discrete mesh data and the distribution of unknown internal self-luminescence light sources, establishing a dynamic sparse regularization target function in every iteration for the equation, and reconstructing a tomography image.

10. The system of claim 9, wherein the optical imaging sub-module comprises a CCD camera.

11. The system of claim 9, wherein the CT imaging sub-module comprises an X-ray emitting source and an X-ray detector which collects data successively.

12. The system of claim 9, wherein the translating table and rotating table are is shared by the optical imaging sub-module and the CT imaging sub-module.

13. The system of claim 9, wherein the optical imaging sub-module and CT imaging sub-module are perpendicular to each other.

14. The system of claim 10, wherein the CCD camera operates in low-temperature state.
